(19) United States
(12) Patent Application Publication
(10) Pub. No.: US 2020/0061014 A1
(43) Pub. Date: Feb. 27, 2020

(54) A PHARMACEUTICAL COMPOSITION COMPRISING RACECADOTRL AND PROCESS FOR PREPARING THE SAME

(71) Applicant: ATHENA PHARMACEUTIQUES SAS, Louveciennes (FR)

(72) Inventors: Mahendra B. Chaudhari, Thane (West), Maharashtra (IN); Amol Y. Chaudhari, Dist. Jalgaon, Maharashtra (IN); Nitin P. Nehete, Dombivili (East), Maharashtra (IN)

(21) Appl. No.: 16/081,634

(22) PCT Filed: Feb. 20, 2018
(86) PCT No.: PCT/IN2018/050085

§ 371 Date: Aug. 31, 2018

(30) Foreign Application Priority Data
Mar. 6, 2017 (IN) 201721007797

(51) Int. Cl. A61K 31/216; A61K 9/16

(52) U.S. Cl. CPC A61K 31/216; A61K 9/1635; A61K 9/1682; A61K 9/1623

(57) ABSTRACT

The present invention relates to a pharmaceutical composition comprising Racecadotril and co-crystallized sugar and a process or preparing the same.

### CROSS REFERENCE TO RELATED APPLICATIONS

This application is a National Stage application of PCT/IN2018/050085, filed Feb. 20, 2018, which claims the benefit of Indian Application No. 201721007797, filed Mar. 6, 2017, both of which are incorporated by reference in their entirety herein.

### FIELD OF THE INVENTION

The present invention relates to a pharmaceutical composition comprising Racecadotril and co-crystallized sugar and a process or preparing the same.

### BACKGROUND OF THE INVENTION

Racecadotril (disclosed in EP038758Bl) is an antidiarrheal drug which acts as an enkephalinase inhibitor which acts by reducing the secretion of water and electrolytes into the intestine. Chemically it is (RS)-Benzyl N-[3-(acetylthio)-2-benzylpropanoyl]glycinate with following chemical structure.

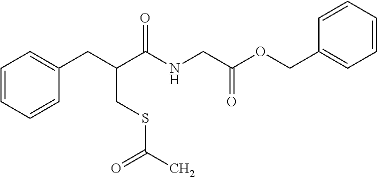 [SMILES: CC(=O)SCC(Cc1ccccc1)C(=O)NCC(=O)OCc1ccccc1]

It is available commercially as Hidrasec® and Tiorfan® in dispersible tablet and sachet dosage forms. Racecadotril being a hydrophobic substance represents difficulties in preparing a suspension dosage form. Hence, it is formulated as a sachet and dispersible tablet dosage form.

EP1294372 discloses a dry powder composition comprising coated granules comprising Racecadotril in association with a pharmaceutically acceptable carrier.

EP2749270 discloses a dispersible tablet comprising Racecadotril whose taste is masked wherein the taste is masked by coating with an acrylic acid polymer or a cellulose polymer by wet granulation method without being mixed with any excipient.

In the existing dosage forms, the drug Racecadotril is coated, although there is no requirement for the same in terms of good palatability, sensitivity to hydrolysis or other environmental conditions, and product appearance. Specifically, racecadotril, when mixed with sweetening agents does not need any coating to show a good palatability. Further, the drug is not sensitive to hydrolysis or any other in vivo degradation process or environmental conditions like moisture and temperature. Also, there is no necessity of any coating to improve the product appearance as well.

Moreover, coated formulations as disclosed in the available dosage forms and disclosures come with additional process requirements and limitations. Generally, the coating process is costly and involves an extra step in manufacturing a simple dosage form like sachet. Also, the selection of coating process and the excipient for the same is difficult because of the hydrophobicity of Racecadotril.

Also, generally, the coatings must be stable and strong enough to survive the handling of the formulation, and must follow the fine contours of embossed characters on the formulation. The coated dosage form results into various defects like Picking and sticking (when coating removes a piece of the formulation from the core), Bridging (coating fills in the lettering or logo on the formulation), Erosion, Twinning (wherein two or more units get adhered to each other because of the coating), Peeling and Frosting (the coating peels away), Blistering (rapid evaporation of solvent from the coated formulation) or Orange peel (coating texture that resembles the surface of an orange). The manufacturing hurdles of the coating process and its effects during the shelf-life of the formulation suggest avoiding the coating process unless absolutely necessary.

Accordingly, there is a need for easy process to manufacture a sachet dosage form for Racecadotril. Also, the process should be easy to develop and should not involve an unessential and complex coating process.

### SUMMARY OF THE INVENTION

To achieve the foregoing and other objects and needs, the present invention provides a pharmaceutical composition comprising Racecadotril and co-crystallized sugar and a process for preparing the same.

In an embodiment, the present invention provides a pharmaceutical composition comprising Racecadotril, co-crystallized sugar and pharmaceutically acceptable inert excipients, wherein the ratio of Racecadotril to co-crystallized sugar is from about 1:10 to about 1:150.

In another embodiment, the present invention provides pharmaceutical composition comprising:
about 0.5% to about 2% w/w of Racecadotril;
about 40% to about 99% w/w of co-crystallized sugar;
about 0.1% to about 5% w/w of disintegrant;
about 0.05% to about 2% w/w of glidant.

In a further embodiment, the present invention provides a process for preparing a pharmaceutical composition comprising Racecadotril, the process comprising the steps of:
(a) Sifting Racecadotril, a portion of co-crystallized sugar and pharmaceutically acceptable inert excipients to form a mixture;
(b) Adding a solvent in polyvinyl pyrrolidone to form a solution or suspension;
(c) Granulating the mixture formed in step (a) with solution or suspension formed in step (b) to form granules;
(d) Drying the granules formed in step (c);
(e) Blending another portion of co-crystallized sugar with silicon dioxide to form intermediate blend;
(f) Mixing the dried granules formed in step (d) and the intermediate blend formed in step (e) to form a final blend;
(g) Filling the final blend formed in step (f) in a suitable sized sachet.

### BRIEF DESCRIPTION OF THE DRAWINGS

The advantages and features of the present invention will become better understood with reference to the following detailed description and claims taken in conjunction with the accompanying drawing, in which:

FIG. 1 illustrates a process flow for preparation of a pharmaceutical composition comprising Racecadotril, in accordance with an exemplary embodiment of the present invention.

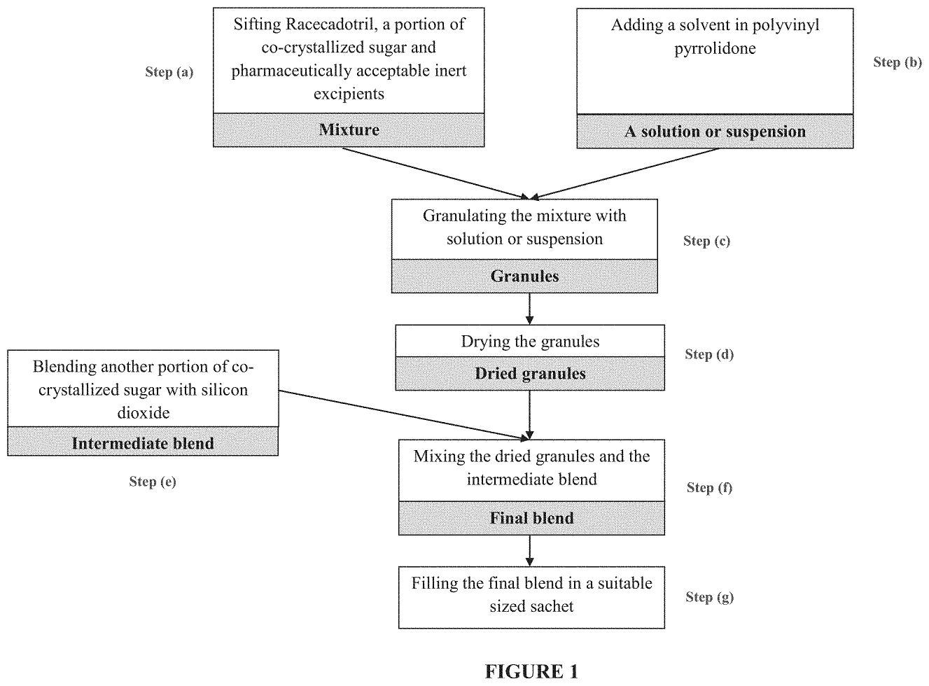

### DETAILED DESCRIPTION OF THE INVENTION

The exemplary embodiments described herein detail for illustrative purposes are subject to many variations in structure and design. It should be emphasized, however, that the present invention is not limited to a pharmaceutical composition comprising Racecadotril and preparation process for the same, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The use of terms “including,” “comprising,” or “having” and variations thereof herein is meant to encompass the items listed thereafter and equivalents thereof as well as additional items. Further, the terms, “a” and “an” herein do not denote a limitation of quantity, but rather denote the presence of at least one of the referenced item.

The present invention provides a pharmaceutical composition (hereinafter referred to as the “composition”) comprising Racecadotril, co-crystallized sugar and pharmaceutically acceptable inert excipients. The composition of the present invention is easy to prepare and does not involve any complex steps like coating. The invention specifically relates to a pharmaceutical composition in a sachet dosage form wherein the ratio of Racecadotril to co-crystallized sugar is from about 1:10 to about 1:150.

In an aspect of the invention, the pharmaceutical composition is in the form of solid oral dosage forms. The solid dosage form may be tablet, capsule, pills, sachets, lozenges, granules, powder and the like. Preferably, the pharmaceutical composition is in the form of a sachet.

As used herein, the term Racecadotril includes free base as well as its pharmaceutically acceptable salts, enantiomers, polymorphic forms.

Co-crystallized sugars as used here include any sugar which is prepared by co-crystallization technique. This technique involves spontaneous crystallization of a supersaturated sugar solution and a second ingredient by agitating it while cooling. This process produces an agglomerated sponge-like structure, with vastly increased surface area. The second ingredient is an integral part of the structure's matrix.

The co-crystallized sugar include dextrose, fructose, isomalt, erythritol, hydrogenated isomaltulose, hydrogenated starch hydrolyzates, lactitol, maltitol, mannitol, polydextrose, sorbitol, sucrose, trehelose, xylitol and the like.

One of the preferred co-crystallized sugars is co-crystallized sucrose. The commercially available grades of co-crystallized sugars may be used for the purpose of carrying out the invention.

In an aspect of the invention, the composition of the present invention comprises the ratio of Racecadotril to co-crystallized sugar from about 1:10 to about 1:150. Specifically, the ratio of Racecadotril to co-crystallized sugar is 1:10 or 1:20 or 1:25 or 1:50 or 1:75 or 1:85 or 1:87.5 or 1:90 or 1:91 or 1:92 or 1:93 or 1:94 or 1:95 or 1:96 or 1:97 or 1:98 or 1:99 or 1:100 or 1:105 or 1:110 or 1:120 or 1:125 or 1:150 or 1:180 or 1:198 and the like.

By the term “pharmaceutically acceptable inert excipients”, it is meant any of the components of a pharmaceutical composition other than active ingredients and which are approved by regulatory authorities or are generally regarded as safe for human or animal use.

Examples of pharmaceutically acceptable inert excipients include, but are not limited to diluents, binders, sweetening agent, disintegrants, solvents, lubricant, glidants and flavorants. A combination of excipients may also be used. The amount of excipient(s) employed will depend upon how much active agent is to be used. One excipient can perform more than one function as well.

Diluents include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate and other materials known to one ordinarily skilled in the art and mixtures thereof. Co-crystallized sugars used in the present invention are used as diluents,

Binders include, but are not limited to, starches such as potato starch, wheat starch, corn starch (maize starch); microcrystalline celluloses; celluloses such as hydroxypropyl cellulose, hydroxyethyl cellulose, hydroxypropyl methylcellulose (HPMC), ethyl cellulose, sodium carboxymethylcellulose; natural gums like acacia, alginic acid, guar gum; liquid glucose, dextrin, povidone, syrup, polyethylene oxide, polyvinylpyrrolidone, poly-N-vinyl amide, polyethylene glycol, gelatin, poly propylene glycol, tragacanth and other materials known to one ordinarily skilled in the art and mixtures thereof.

The sweetening agents (also known as sweeteners) include, but are not limited to, aspartame, saccharin sodium, dipotassium glycyrrhizinate, stevia, thaumatin, acesulfame K, sucralose, and other materials known to one ordinarily skilled in the art and mixtures thereof.

The disintegrants include, but are not limited to, starch, croscarmellose sodium, polyvinyl pyrrolidone, sodium starch glycolate and other materials known to one ordinarily skilled in the art and mixtures thereof. Preferably, the disintegrant is polyvinyl pyrrolidone.

Solvents include, but are not limited to purified water, acetone, ethyl alcohol, isopropyl alcohol dichloromethane and other materials known to one ordinarily skilled in the art and mixtures thereof.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil, talc and other materials known to one ordinarily skilled in the art and mixtures thereof.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one ordinarily skilled in the art and mixtures thereof. Preferably, the glidant is colloidal silicon dioxide.

The flavorants include natural and artificial flavors. These flavors include, but are not limited to synthetic flavor oils and flavoring aromatics, and/or oils, oleo resins and extracts derived from plants, leaves, flowers, fruits, etc., and other materials known to one ordinarily skilled in the art and mixtures thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors also include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used.

The present invention further relates to a pharmaceutical composition comprising:
about 0.5% to about 2% w/w of Racecadotril;
about 40% to about 99% w/w of co-crystallized sugar;
about 0.1% to about 5% w/w of disintegrant;
about 0.05% to about 2% w/w of glidant.

The present invention further relates to a process for preparing a pharmaceutical composition comprising Racecadotril. Conventional processes for preparing the compositions like wet granulation, dry granulation and direct compression can be used.

In an embodiment, the present invention related to a process for preparing a pharmaceutical composition comprising Racecadotril, the process comprising the steps of:
(a) Sifting Racecadotril, a portion of co-crystallized sugar and pharmaceutically acceptable inert excipients to form a mixture;
(b) Adding a solvent in polyvinyl pyrrolidone to form a solution or suspension;
(c) Granulating the mixture formed in step (a) with solution or suspension formed in step (b) to form granules;
(d) Drying the granules formed in step (c);
(e) Blending another portion of co-crystallized sugar with silicon dioxide to form intermediate blend;
(f) Mixing the dried granules formed in step (d) and the intermediate blend formed in step (e) to form a final blend;
(g) Filling the final blend formed in step (f) in a suitable sized sachet.

Mixing of the excipients can be performed in a conventional device used for mixing of powders, such as for example motionless (passive) mixers, fluidized bed, diffusion, bi-conic diffusion, uniconic, biconic, turbular, cubic, planetary, Y-, V-shaped, and low shear or high shear mixers.

The granulation operation may be performed using apparatus such as, for example, a fluidized-bed granulator, an agitation granulator, a biaxial granulator, or the like.

Generally, the granulated mixture is preferably dried after granulation in step (c) in any pharmaceutical acceptable manner. Drying of the granulate for example can be performed in one of the following ways: trays, fluid bed, and microwave assist/vacuum/gas stripping (one pot processing).

The granulation may be carried out by suitable aqueous or non-aqueous solvents.

The pharmaceutical composition according to the invention provides a further advantage of not having to coat the drug Racecadotril. As described above, there is no requirement for the coating Racecadotril for achieving good palatability, sensitivity to hydrolysis or other environmental conditions, and product appearance. Accordingly, the process of the invention avoids the process of coating, thereby avoiding additional coating process.

The description of the present invention of pharmaceutical composition comprising Racecadotril is further illustrated by the following non-limiting example. However, a person skilled in the art would recognize that, the specific example is intended to illustrate, not limit, the scope of the present invention.

### EXAMPLES

### Example 1: Pharmaceutical Composition According to the Present Invention

TABLE 1
Pharmaceutical composition comprising Racecadotril

| Ingredients | % W/W | 10 mg/sachet | 30 mg/sachet |
|---|---|---|---|
| Racecadotril | 1.00 | 10.00 | 30.00 |
| Co-crystallized sucrose | 41.50 | 415.00 | 1245.00 |
| Polyvinyl pyrrolidone | 0.15 | 0.50 | 15.00 |
| Purified water | Q.S | Q.S | Q.S |
| Co-crystallized sucrose | 57.25 | 572.50 | 1717.50 |
| Colloidal silicon dioxide | 0.10 | 1.00 | 3.00 |
| Total | 100 | 1000 | 3000 |

### Manufacturing Process:

### 1. Preparation of Drug Granules:

Co-crystallized sucrose was milled in a suitable mill. It was sifted with intermediate addition of Racecadotril & colloidal silicon dioxide in a suitable sifter. The sifted mass was added in a mixer and mixed.
Purified water was added in polyvinyl pyrrolidone to form a suspension. The binder suspension was added to the dry mix in a suitable granulator. The wet mass was dried in a suitable dryer. The dried granules were sifted and milled. The dried granules were transferred to a suitable blender and mixed thoroughly.

### 2. Preparation of Bulk Granules:

Another portion of co-crystallized sucrose was milled in a suitable mill. Racecadotril and milled co-crystallized sucrose were sifted. The sifted mass was blended properly. Blended mass of drug granules materials was transferred in to the blender containing intermediate blend and mixed properly. The blended material in the above step was sifted again and mixed properly.

### 3. Sachets Filling:

Racecadotril bulk granules were filled in a suitable sachet.
The packing details were as follows:

TABLE 2
Packing details for pharmaceutical
composition comprising Racecadotril

| | | | |
|---|---|---|---|
| SN | PARAMETERS | STANDARD | LIMITS |

Racecadotril sachets 10 mg

| | | |
|---|---|---|
| 1 | Appearance | White to off-white granules filled |
| | | in heat sealed aluminum sachets |

| | | | |
|---|---|---|---|
| 2 | Average fill weight | 1000.00 mg | 1000 mg ± 5% |
| | | | (950-1050 mg) |

Racecadotril sachets 30 mg

| | | |
|---|---|---|
| 1 | Appearance | White to off-white granules filled |
| | | in heat sealed aluminum sachets |

| | | | |
|---|---|---|---|
| 2 | Individual Sachet | 3000 mg | 3000 mg ± 5% |
| | weight | | (2850-3150 mg) |

We claim:

1. A pharmaceutical composition comprising Racecadotril, co-crystallized sugar and pharmaceutically acceptable inert excipients, wherein the ratio of Racecadotril to co-crystallized sugar is from about 1:10 to about 1:150.

2. The pharmaceutical composition according to claim 1, wherein the composition is uncoated.

3. The pharmaceutical composition according to claim 1, wherein the pharmaceutically acceptable inert excipients comprise diluents, binders, sweetening agent, disintegrants, solvents, lubricant, glidants and flavorants.

4. The pharmaceutical composition according to claim 1, wherein the co-crystallized sugar is co-crystallized sucrose.

5. A pharmaceutical composition comprising:
about 0.5% to about 2% w/w of Racecadotril;
about 40% to about 99% w/w of co-crystallized sugar;
about 0.1% to about 5% w/w of disintegrant;
about 0.05% to about 2% w/w of glidant.

6. The pharmaceutical composition according to claim 5, wherein the composition is a solid dosage form.

7. The pharmaceutical composition according to claim 5, wherein the composition is in the form of a sachet.

8. The pharmaceutical composition according to claim 5, wherein the co-crystallized sugar is co-crystallized sucrose.

9. The pharmaceutical composition according to claim 5, wherein the disintegrant is polyvinyl pyrrolidone.

10. The pharmaceutical composition according to claim 5, wherein the glidant is colloidal silicon dioxide.

11. A process for preparing a pharmaceutical composition comprising Racecadotril, the process comprising the steps of:
(a) Sifting Racecadotril, a portion of co-crystallized sugar and pharmaceutically acceptable inert excipients to form a mixture;
(b) Adding a solvent in polyvinyl pyrrolidone to form a solution or suspension;
(c) Granulating the mixture formed in step (a) with solution or suspension formed in step (b) to form granules;
(d) Drying the granules formed in step (c);
(e) Blending another portion of co-crystallized sugar with silicon dioxide to form intermediate blend;
(f) Mixing the dried granules formed in step (d) and the intermediate blend formed in step (e) to form a final blend;
(g) Filling the final blend formed in step (f) in a suitable sized sachet.

12. The process according to claim 11, wherein the granulation process is aqueous granulation.

13. The process according to claim 11, wherein the granulation process is non-aqueous granulation.
